(19) United States
(12) Patent Application Publication
(10) Pub. No.: US 2026/0137270 A1
(43) Pub. Date: May 21, 2026

(54) CapsuleProbe - Application and Method of Capsule Endoscopy in Cervical Examination

(71) Applicants: Emerald Wang, Carmel, IN (US); Chi Wang, Carmel, IN (US); Katherine Wang, Carmel, IN (US)

(72) Inventors: Emerald Wang, Carmel, IN (US); Chi Wang, Carmel, IN (US); Katherine Wang, Carmel, IN (US)

(21) Appl. No.: 19/414,288

(22) Filed: Dec. 10, 2025

Related U.S. Application Data
(63) Continuation-in-part of application No. 18/822,086, filed on Aug. 31, 2024.

(51) Int. Cl. A61B 1/303; A61B 1/00; A61B 1/04

(52) U.S. Cl. CPC A61B 1/303; A61B 1/00096; A61B 1/00101; A61B 1/00108; A61B 1/041

(57) ABSTRACT

The invention relates to a method and an apparatus, termed the “CapsuleProbe”, adapted from the capsule endoscopy technology for cervical and/or vaginal examinations. The CapsuleProbe features a capsule endoscope enclosed in an elongated cylindrical housing designed for easy insertion into the vaginal canal. This device provides a minimally invasive and user-friendly option for patients as well as enhanced visualization of the cervix for healthcare providers. With its substantially minimized dimensions comparing to existing medical devices for cervical and/or vaginal examinations, this device improves feasibility, accessibility, and patient comfort significantly, particularly for postmenopausal women and those suffering from vaginal atrophy, by reducing or eliminating procedure-related pain and/or bleeding from the tearing of vaginal walls caused by existing medical devices used for cervical and/or vaginal examinations.

### CROSS-REFERENCE TO RELATED APPLICATIONS

This application is a continuation-in-part of U.S. application Ser. No. 18/822,086, filed Aug. 31, 2024, the entire disclosure of which is incorporated herein by reference. This application claims the benefit of U.S. application Ser. No. 18/822,086 filed Aug. 31, 2024.

### FIELD OF THE INVENTION

The present invention relates to the field of medical diagnostic devices, specifically to a novel application and method of capsule endoscopy for cervical examination. This invention pertains to a specially configured “CapsuleProbe”, intended for use in gynecological diagnostics.

### BACKGROUND OF THE INVENTION AND PRIOR ART

Capsule endoscopy, using a standalone pill-size camera, has revolutionized the examination of the gastrointestinal tract, allowing for minimally invasive diagnostics. However, current technologies have not been adapted for gynecological applications, particularly for cervical examinations. Traditional methods of cervical examination, such as Pap smears and colposcopy, though effective, can be painful and even physically damaging for postmenopausal women and patients with vaginal atrophy. These challenges can discourage or even prevent affected women from undergoing regular or timely cervical exam, therefore increase their risks of missing early detection of cervical cancer.

### Prevalence of Vaginal Atrophy and the Need for Improved Examination Methods

Vaginal atrophy is a common condition among postmenopausal women, affecting approximately 50% of this population. This condition, characterized by thinning, drying, and inflammation of the vaginal walls, is associated with decreased estrogen levels during menopause. Vaginal atrophy can lead to symptoms such as discomfort, pain, and increased vulnerability to physical tearing, particularly during vaginal examinations.

The population of women aged 50 and older is significant in the US including Boomers and a large portion of Gen-X, with global trends indicating a significant rise in the number of postmenopausal women in the coming decades. As a result, there is a growing need for medical devices and examination methods that are tailored to the unique physiological changes that occur in this demographic.

### Challenges With Traditional Vaginal Examinations

The Vaginal Speculum 100A FIG. 5, a widely used instrument for gynecological examinations, presents several challenges, especially for postmenopausal women. The speculum is typically large, hard, and sharp-edged, which can cause significant discomfort, pain, and even physical tearing of the vaginal walls during insertion and examination. These issues are exacerbated in women with vaginal atrophy, where the vaginal tissue is more delicate and prone to injury.

Another less common but more expensive procedure is transvaginal ultrasound. Again, for postmenopausal women coping with vaginal atrophy, this procedure can be painful and physically damaging since the size of the transducer probe is as large as a regular vaginal speculum 100B FIG. 6. It can cause tearing of vaginal walls and sometimes fail to be pushed into the vaginal canal due to its large size for petite women.

Given these challenges, there is an urgent need for alternative examination methods and devices that are less invasive and more comfortable for patients. The present invention addresses this need by introducing a novel standalone miniature device with an enclosed capsule endoscope designed specifically for cervical examination. This device allows for enhanced visualization and examination of the cervix in a minimally invasive manner with much improved ease of use and patient comfort, particularly for women suffering from vaginal atrophy.

### SUMMARY OF THE INVENTION

The present invention provides a novel method and apparatus for cervical examination utilizing a pill-sized capsule endoscope enclosed in a specially configured housing, referred to as “CapsuleProbe”. The CapsuleProbe is a slim cylindrical housing unit with a standalone pill-size capsule endoscope mounted inside of the housing unit that facilitates easy insertion into the vaginal canal, positioning the capsule near the cervix for optimal imaging, and simple retrieval after the examination. By adapting a standalone pill-size capsule endoscope for gynecological purposes, and with a substantially minimized dimensional size, the present invention can significantly advance diagnostics in cervical health and cancer prevention by providing a much more comfortable and drastically less invasive option for patients.

The invention includes the following key components:
1. Capsule endoscope: An existing standalone pill-size capsule endoscope.
2. Elongated Cylindrical Housing Unit: A housing unit with a slim cylindrical housing body configured to correspond to the size of a regular tampon, designed to hold a pill-size capsule endoscope at the distal end of the housing unit enclosed under a transparent dome cover, to insert into the vaginal canal and place it next to the cervix. The housing body is made from a biocompatible material and is shaped round and smooth to allow for easy and comfortable insertion into the vaginal canal. The housing body is made of the same disposable tampon housing materials of biocompatible plastic or composite-fibers, or of medical-grade plastic or silicone for repeated use after sterilization, if deemed economical and practical. The transparent dome cover is made of clear medical-grade plastic.

### Objects of the Invention

1. Patient Comfort: To provide a method for cervical examination that is much less invasive and more comfortable for patients compared to traditional methods, particularly for women with vaginal atrophy.
2. Enhanced Visualization: To offer a device that provides enhanced visualization of the cervix, leading to improved diagnostic capabilities.
3. Ease of Use: To design a device that can be easily inserted and retrieved by patients or healthcare providers.
4. Minimally Invasive: To create a device with significantly minimized dimensions that reduces tearing of vaginal walls.

### BRIEF DESCRIPTION OF THE DRAWINGS

1. FIG. 1 depicts the general shape, look, and the camera-head of a capsule endoscope.

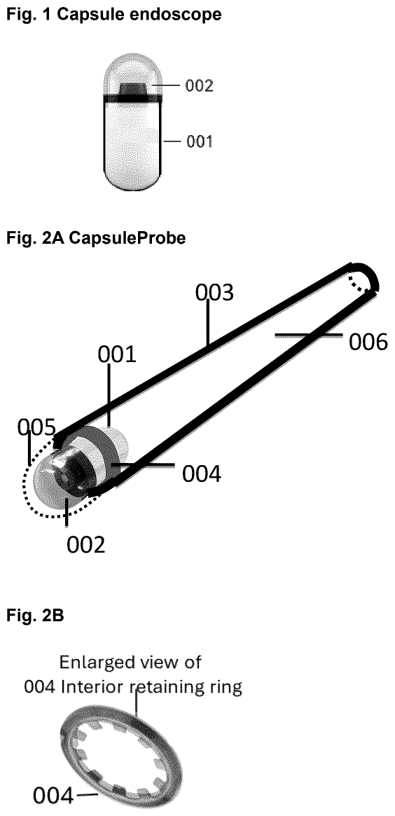

2. FIG. 2A-2C illustrates perspective views of the device and its components.

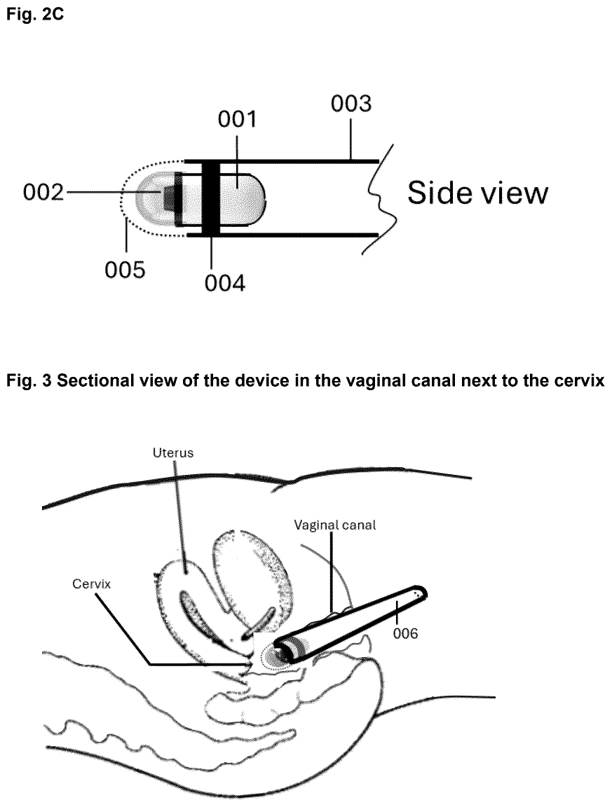

3. FIG. 3 shows a side section view of the device in use, placed inside of vaginal canal and next to the cervix.

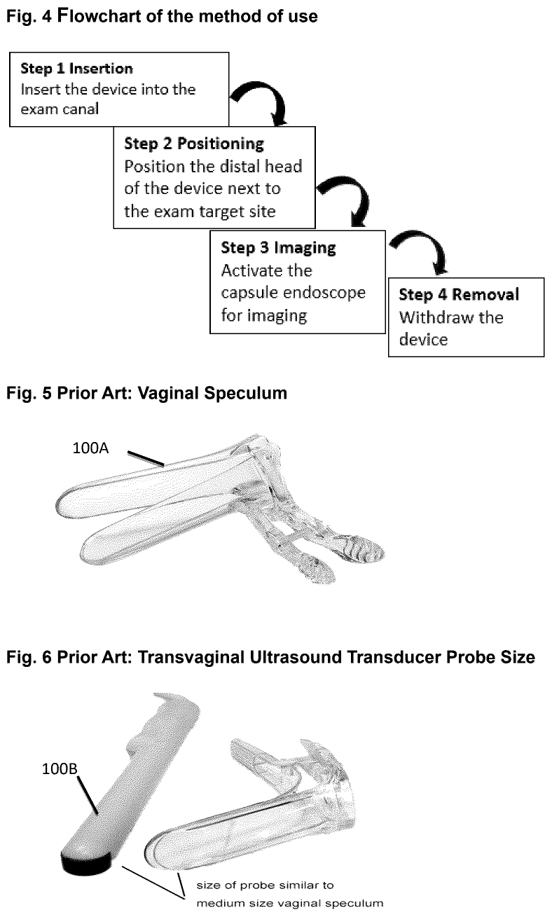

4. FIG. 4 is a flowchart for the method of use.

5. FIG. 5 depicts a Vaginal Speculum referenced in the Background/Prior Art section.

6. FIG. 6 illustrates the size of a Transvaginal Ultrasound Transducer Probe as similar to a Vaginal Speculum, referenced in the Background/Prior Art section.

### DETAILED DESCRIPTION OF THE INVENTION

### Capsuleprobe

The CapsuleProbe is based on existing technology of capsule endoscope FIG. 1 but adapted for gynecological use. The camera head of the capsule scope [002 FIG. 1] is equipped with a high-resolution camera and light source, allowing for detailed imaging of the cervix in this application. The capsule itself is powered by a small internal battery and can take and transmit real-time video footage or pictures wirelessly to an external recorder or monitor, where a healthcare provider can assess the condition of the cervix.

The elongated slim cylindrical housing unit forms the body of the CapsuleProbe FIG. 2A-C. It holds and places the capsule endoscope [001 FIG. 2A-2C] through the vaginal canal to reach the cervix. The housing unit [003 FIG. 2A-2C] is configured to correspond to the size of a regular tampon and is made from a biocompatible material, such as existing disposable tampon housing materials of biocompatible plastic or composite fibers. It can also be made from reusable materials such as medical-grade plastics or flexible materials such as medical-grade silicone for further enhanced patient comfort. The exterior of the housing is smooth and rounded, ensuring easy and comfortable insertion.

The housing features an interior retaining ring [004 FIG. 2A-2C] made from strong and elastic materials such as silicone or rubber. It is fixed to the interior wall of the housing by strong medical-grade glue. The retaining ring securely holds the capsule endoscope with its camera head [002 FIG. 2A-2C] facing outward at the distal end of the housing. The camera head is enclosed under a transparent dome cover [005 FIG. 2A-2C] made from clear medical-grade plastic. The dome cover is attached to the housing body by strong medical-grade glue, or male-female threads. The dome cover prevents the capsule from getting trapped inside of the vaginal canal in the event of it becoming loose from the retaining ring.

Using CapsuleProbe is as easy as placing a tampon. Once inserted into the vaginal canal, the CapsuleProbe positions the enclosed capsule endoscope next to the cervix FIG. 3. After the imaging is finished, a patient or healthcare provider can easily retrieve the CapsuleProbe.

### Method of Use FIG. 4

The method of use for the CapsuleProbe in cervical examination is as follows:
1. Insertion: A patient or healthcare provider inserts the CapsuleProbe into the vaginal canal.
2. Positioning: Place the CapsuleProbe to position its distal end next to the cervix.
3. Imaging: Activate the capsule endoscope for imaging.
4. Removal: After the imaging is completed, withdraw the CapsuleProbe.

### Definition of “Regular Tampon”

As used herein, the term “regular tampon” refers to a class of intravaginal articles, tampons, corresponding to the “Regular” absorbency category, as the term is used in the context of menstrual tampons and defined by an absorbency capacity of approximately 6-9 grams as measured by standardized test methods.

In industry practice, tampons corresponding to the regular absorbency category are typically associated with an external diameter of approximately 11-13 millimeters and a length of approximately 43-52 millimeters, reflecting a dimensional envelope suitable for self-insertion.

For purposes of this disclosure, a housing unit described as corresponding to “the size of a regular tampon” denotes a housing geometry corresponding to this absorbency-defined class and associated dimensional envelope. Such a housing geometry is suitable for retaining a pill-size capsule endoscope and associated retaining structures and for placing the capsule next to the cervix in a minimally invasive manner that reduces insertion-related tissue trauma.

What is claimed is:

1. A medical device for cervical examination comprising:
a standalone capsule endoscope including a camera, a light source, an internal power source, and wireless transmission capability;
a smooth cylindrical housing unit made from biocompatible material and with the size corresponding to a regular tampon;
an internal retaining ring positioned within the cylindrical housing unit, configured to securely hold the capsule endoscope with the camera oriented outwardly towards the distal end of the housing unit; and
a transparent dome cover attached to the distal end of the housing unit, configured to safeguard the capsule endoscope from detachment and minimize insertion trauma.

2. The medical device of claim 1, wherein the cylindrical housing unit is composed of the same disposable biocompatible plastics used for conventional tampon housing, or medical-grade plastics or silicone for sterilization and reuse.

3. The medical device of claim 1, wherein the internal retaining ring is made from strong and elastic materials of silicone or rubber.

4. The medical device of claim 1, wherein the internal retaining ring is fixed to the interior wall of the housing unit by strong medical-grade glue.

5. The medical device of claim 1, wherein the transparent dome cover is securely fastened to the housing unit by strong medical-grade glue or male-female threads.

6. A method of cervical examination using the medical device of claim 1 comprising:
(a) inserting the medical device into the vaginal canal by a patient or healthcare provider;
(b) positioning the transparent dome cover next to the cervix;
(c) activating the capsule endoscope for imaging; and
(d) retrieving the device after imaging is completed.
